(19) United States
(12) Patent Application Publication
(10) Pub. No.: US 2020/0353225 A1
(43) Pub. Date: Nov. 12, 2020

(54) RAPID EXCHANGE SYSTEM AND METHODS FOR USE

(71) Applicant: SANFORD HEALTH, Sioux Falls, SD (US)

(72) Inventor: Patrick W. KELLY, Sioux Falls, SD (US)

(21) Appl. No.: 16/640,608

(22) PCT Filed: Aug. 30, 2018
(86) PCT No.: PCT/US2018/048762

§ 371 Date: Feb. 20, 2020

Related U.S. Application Data
(60) Provisional application No. 62/552,233, filed on Aug. 30, 2017.

(51) Int. Cl. A61M 25/09; A61M 25/00

(52) U.S. Cl. CPC A61M 25/09041; A61M 25/007; A61M 2025/0183; A61M 2025/0079; A61M 25/0074

(57) ABSTRACT

The present disclosure provides an apparatus including a catheter having a first end and a second end and defining a lumen extending from the first end to the second end. The apparatus also includes one or more openings positioned in a side wall of the catheter between the first end and the second end of the catheter. The apparatus also includes one or more coverings positioned over the one or more openings. A diverter is configured to contact a first covering of the one or more coverings to transition the first covering from a first position to a second position where the first covering projects away from a side wall of the catheter to at least partially obstruct the lumen of the catheter such that the first opening of the one or more openings is accessible via the lumen.

### RELATED APPLICATIONS

This application claims the benefit of priority to U.S. Provisional Application No. 62/552,233 entitled “Rapid Exchange System and Methods for Use,” filed on Aug. 30, 2017, the contents of which are hereby incorporated by reference in its entirety.

### BACKGROUND THE INVENTION

As the distance between the point of ex vivo arterial access and the point of in vivo treatment increases, the required catheter support needed to coaxially deliver an endovascular treatment also increases. Currently, this challenge is overcome by stiff wires and added support of long sheaths. The challenge of this solution is that the long sheaths are required to be the coaxial line for delivery of therapies. As the length of the sheath increases, operator control and manipulation of the sheath becomes more challenging thereby adding to the complexity of cases. Such added complexity increases procedure time and indirectly increases the likelihood of complication for the procedure.

In an attempt to solve this problem, rapid exchange systems have been utilized in coronary interventions. Rapid exchange systems permit a very rapid, atraumatic means of exchanging one balloon catheter or other device for a balloon dilatation catheter or other device. However, rapid exchange systems have not been used as frequently in peripheral vasculature interventions for several reasons. The first reason is that variable guide wire sizes are used in peripheral vasculature interventions. In the coronary intervention market, 0.014 guide wires are almost universally used. However, in the peripheral vasculature interventions market, 0.014, 0.018 and 0.035 guide wires are used. Also, because of the long distances to navigate to the treatment vessel and the long length of the diseased treatment segment, catheter support is paramount in peripheral vasculature interventions. Yet, traditional rapid exchange systems sacrifice support for convenience.

### SUMMARY OF THE INVENTION

The present disclosure relates to a device that advantageously permits rapid exchange when desirable but full over-the-wire support when needed.

Thus, in a first aspect, the present disclosure provides an apparatus that includes: (a) a catheter having a first end and a second end, wherein the catheter defines a lumen extending from the first end to the second end, (b) one or more openings positioned in a side wall of the catheter between the first end and the second end of the catheter, and (c) one or more coverings positioned over the one or more openings in the catheter in a first position, wherein a diverter is configured to contact a first covering of the one or more coverings such that the first covering transitions from the first position where the first covering is positioned over a first opening of the one or more openings to a second position where the first covering projects away from a side wall of the catheter towards a midline of the lumen to at least partially obstruct the lumen of the catheter such that the first opening of the one or more openings is accessible via the lumen.

In a second aspect, the present disclosure provides an apparatus that includes: (a) a catheter having a first end and a second end, wherein the catheter defines a lumen extending from the first end to the second end, (b) one or more openings positioned in a side wall of the catheter between the first end and the second end of the catheter; and (c) a liner having a deformable segment positioned in the lumen, wherein the deformable segment is positioned at least partially over a first opening of the one or more openings when the deformable segment is in a first position, and wherein the deformable segment is configured to project away from the side wall of the catheter towards a midline of the lumen in a second position to at least partially obstruct the lumen of the catheter such that the first opening is accessible via the lumen.

In a third aspect, the present disclosure provides a method that includes: (a) inserting a diverter through the first opening of the apparatus according to the first aspect from an exterior of the catheter and into the lumen of the catheter such that the first covering projects away from the side wall of the catheter towards a midline of the lumen to at least partially obstruct the lumen of the catheter, (b) introducing the apparatus into a target lumen via arterial access, and (c) introducing a guide wire into the lumen of the catheter such that the guide wire contacts the first covering and is thereby directed through the first opening in the catheter.

In a fourth aspect, the present disclosure provides a method that includes: (a) transitioning the deformable member of the apparatus according to the second aspect from the first position to the second position such that the deformable member projects away from the side wall of the catheter towards a midline of the lumen to at least partially obstruct the lumen of the catheter, (b) introducing the apparatus into a target lumen via arterial access, and (c) introducing a guide wire into the lumen of the catheter such that the guide wire contacts the deformable segment and is thereby directed through the first opening in the catheter.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is a side cross-section view of an example apparatus in a first position, according to an example embodiment.

FIG. 2 is a side cross-section view of the apparatus of FIG. 1 in a second position, according to an example embodiment.

FIG. 3 is a side cross-section view of another example apparatus in a first position, according to an example embodiment.

FIG. 4 is a front cross-section view of the apparatus of FIG. 3 taken along line A:A in a first position, according to an example embodiment.

FIG. 5 is a side cross-section view of the apparatus of FIG. 3 in a second position, according to an example embodiment.

FIG. 6 is a front cross-section view of the apparatus of FIG. 5 taken along line B:B in a second position, according to an example embodiment.

FIG. 7 is a flow chart depicting functions that can be carried out in accordance with example embodiments of the disclosed methods.

FIG. 8 is another flow chart depicting functions that can be carried out in accordance with example embodiments of the disclosed methods.

### DETAILED DESCRIPTION OF THE INVENTION

The description of the different advantageous arrangements are presented for purposes of illustration and description, and are not intended to be exhaustive or limited to the examples in the form disclosed. Many modifications and variations will be apparent to those of ordinary skill in the art. Further, different examples may provide different advantages as compared to other advantageous examples. The example or examples selected are chosen and described in order to best explain the principles of the examples, the practical application, and to enable others of ordinary skill in the art to understand the disclosure for various examples with various modifications as are suited to the particular use contemplated.

As used herein, with respect to measurements, “about” means +/−5%.

As used herein, “substantially parallel” means +/−5%.

As used herein, “coupled” means associated directly, as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” denotes existing characteristics of an apparatus, element, and method which enable the apparatus, element, and method to perform the specified function without further modification. For purposes of this disclosure, an apparatus, element, and method described as being “configured to” perform a particular function may additionally or alternatively be described as being “adapted to” and/or as being “operative to” perform that function.

As used herein, a “catheter” is an apparatus that is connected to a deployment mechanism and is configured to house a medical device that can be delivered over a guide wire. The catheter may include a guide wire lumen for over-the-wire guidance and may be used for delivering the medical device to a target lumen.

As used herein, a “guide wire” is an elongated cable comprised of one or more biocompatible materials including metals and polymers. Guide wires may be used for selecting target lumens and guiding catheters to target deployment locations. Guide wires are typically defined as wires used independently of other devices that do not come as part of an assembly.

As used herein, “lumen” refers to a passage within an arterial or tubular structure, such as the pulmonary arteries or a passage within the tubular housings or catheters through which a guide wire may be disposed.

As used herein, “first end” refers to the end of the apparatus that will be a “distal end” closer to the target location upon deployment in vivo.

As used herein, “second end” refers to the end of the apparatus that will be a “proximal end” closer to the operator upon deployment in vivo.

As used herein, “opening” means a diversion point in the catheter that may or may not be in free communication with the exterior of the catheter.

As used herein, all references to the “first opening” and its corresponding structure applies to all subsequent additional openings.

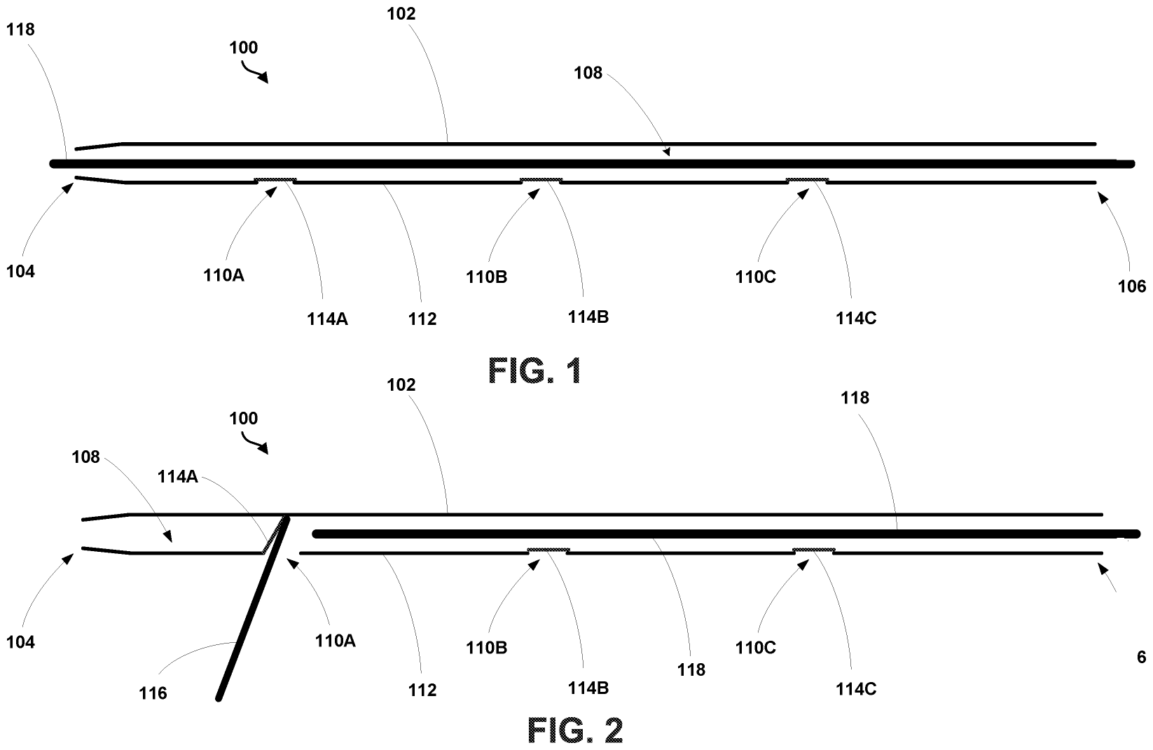

With reference to the Figures, FIG. 1 illustrates an example apparatus 100 that includes a catheter 102 having a first end 104 and a second end 106. The catheter 102 defines a lumen 108 extending from the first end 104 to the second end 106. The apparatus 100 further includes one or more openings 110 positioned in a side wall 112 of the catheter 100 between the first end 104 and the second end 106 of the catheter 102. The apparatus 100 further includes one or more coverings 114A-114C positioned over the one or more openings 110A-10C in a first position, as shown in FIG. 1, such that the lumen 108 is not accessible via the one or more openings 110A-110C from an exterior of the catheter 102 when the one or more coverings 114A-114C are in the first position. The one or more coverings 114A-114C may comprise a perforated material positioned over the one or more openings 110A-110C in the catheter 102. In such an example, the perforated material may comprise a gel, a mesh, a cloth, or polytetrafluoroethylene (PTFE). As such, the perforated material may be configured to cover the one or more openings 110A-110C in the first position, and may be further configured to enable a diverter 116 to be positioned through one of the one or more openings 110A-110C. In another example, the one or more openings 110A-110C may be open to the exterior of the catheter 102 in the first position. In another example, the one or more coverings 114A-114C may comprise a one-way valve, hinged opening, or other cover configured to transition from a closed state (i.e., first position) to an opened state (i.e., second position) in response to receiving the diverter 116.

The apparatus 100 is configured to receive a diverter 116 through a first opening 110A of the one or more openings 110A-110C. The diverter 116 is configured to contact a first covering 114A of the one or more coverings 114A-114C such that the first covering 114A transitions from the first position where the first covering 114A is positioned over a first opening 110A of the one or more openings 110A-110C to a second position where the first covering 114A projects away from the side wall 112 of the catheter 102 towards a midline of the lumen 108 to at least partially obstruct the lumen 108 of the catheter 102, such that the first opening 110A is accessible via the lumen 108, as shown in FIG. 2. The diverter 116 may include an elongated shaft, such as a blunted needle as one non-limiting example, that is positionable through the first opening 110A from an exterior of the catheter 102 and into the lumen 108 of the catheter 102. The diverter 116 may be inserted into the first opening 110A to transition the first covering 114A from the first position to the second position prior to deployment of the apparatus 100. As such, the diverter 116 may be inserted into the first opening 110A when the apparatus 100 is er vivo. Each of the one or more coverings 114A-114C may be configured to be permanently deformed to the second position in response to a force applied by the diverter 116.

The apparatus may further include a guide wire 118 positioned in the lumen 108 in the first position. In one example, as shown in FIG. 1, the guide wire 118 may comprise a through wire extending from the first end 104 of the catheter 102 to the second end 106 of the catheter 102. In another example, as shown in FIG. 2, the guide wire 118 is configured to contact the first covering 114A when the first covering 114A is in the second position such that the guide wire 118 is thereby directed through the first opening 110A in the side wall 112 of the catheter 102. As such, the guide wire 118 is able to more easily access arterial branches or side branches out of the one or more openings 110A-110C when the one or more coverings 114A-114C are transitioned to the second position.

In one example, the first opening 110A is positioned between about 10 mm to about 240 mm from the first end 104 of the catheter 102. In another example, the apparatus 100 further includes a second opening 110B of the one or more openings 110A-110C positioned between the first opening 110A and the second end 106 of the catheter 102. In such an example, the second opening 110B may be configured to receive the diverter 116 such that the second covering 114B projects away from the side wall 112 of the catheter 102 towards the midline of the lumen 108 to at least partially obstruct the lumen 108 of the catheter 102 such that the second opening 110B is accessible via the lumen 108. In another example, a second diverter (not shown) may be used in the second opening 110B in addition to the diverter 116 used in the first opening 110A. The first opening 110A may be positioned between about 10 mm to about 50 mm from the first end 104 of the catheter 102, and the second opening 110B may be positioned between about 30 mm to about 240 mm from the first end 104 of the catheter 102.

In yet another example, the apparatus 100 further includes a third opening 110C of the one or more openings 110A-110C positioned between the second opening 110B and the second end 106 of the catheter 102. In such an example, the third opening 110C may be configured to receive the diverter 116 such that the third covering 114C projects away from the side wall 112 of the catheter 102 towards the midline of the lumen 108 to at least partially obstruct the lumen 108 of the catheter 102 such that the third opening 110C is accessible via the lumen 108. In another example, a third diverter (not shown) may be used in the third opening 110C in addition to a second diverter used in the second opening 110B and the diverter 116 used in the first opening 110A. The first opening 110A may be positioned between about 10 mm to about 50 mm from the first end 104 of the catheter 102, the second opening 110B may be positioned between about 30 mm to about 100 mm from the first end 104 of the catheter 102, and the third opening 110C may be positioned between about 50 mm to about 240 mm from the first end 104 of the catheter 102.

In one example, each of the first opening 110A, the second opening 110B, and the third opening 110C have the same diameter. In another example, each of the first opening 110A, the second opening 110B, and the third opening 110C have different diameters. The first opening 110A, the second opening 110B, and the third opening 110C gives a user the ability to determine which one (if any) of the coverings 114A-114C should be transitioned from the first position to the second position prior to inserting the apparatus 100 into a patient, depending on the particular use case.

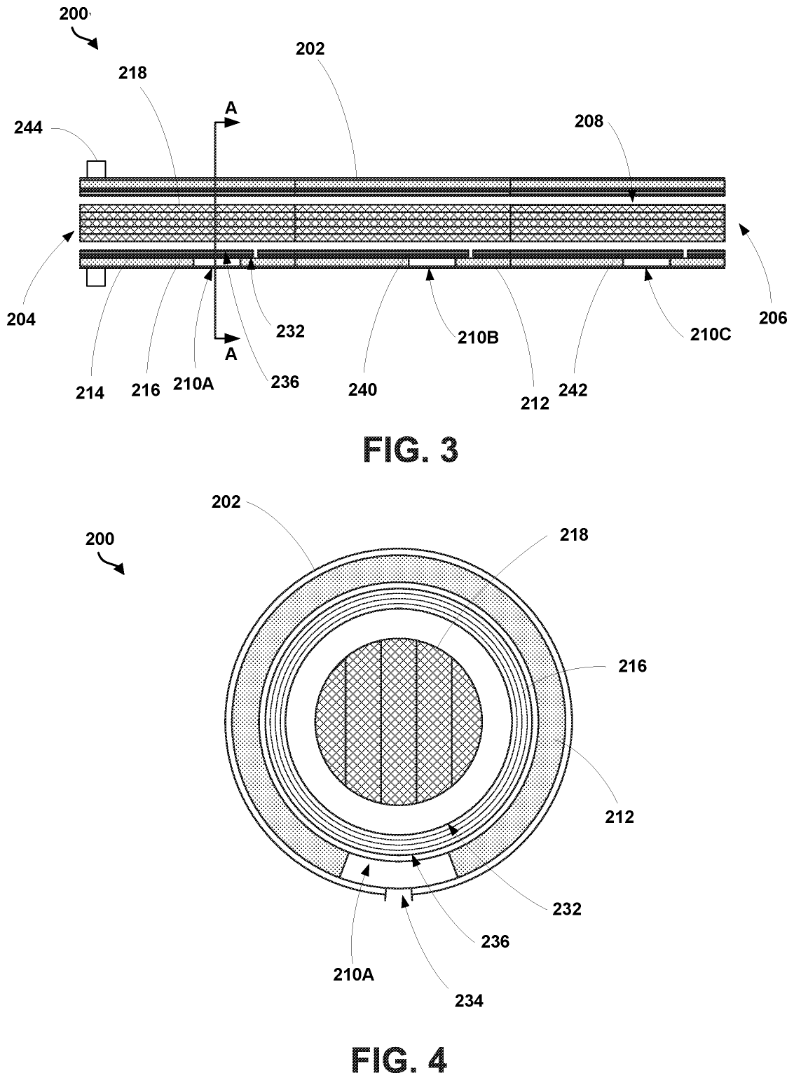

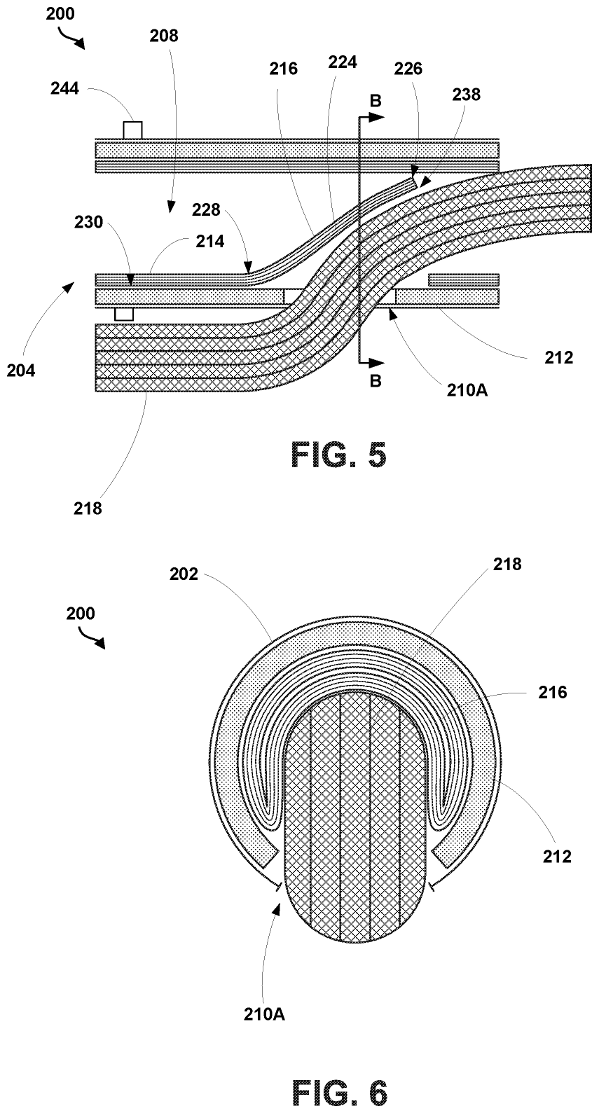

FIGS. 3-6 illustrate another apparatus 200, according to an example embodiment. As shown in FIG. 3, the apparatus 200 includes a catheter 202 having a first end 204 and a second end 206. The catheter 202 defines a lumen 208 extending from the first end 204 to the second end 206. The apparatus 200 includes one or more openings 210A-210C positioned in a side wall 212 of the catheter 202 between the first end 204 and the second end 206 of the catheter 202. The apparatus 200 also includes a liner 214 having a deformable segment 216 positioned in the lumen 208 of the catheter 202. The deformable segment 216 is positioned at least partially over a first opening 210A of the one or more openings 210A-210C in a first position, as shown in FIGS. 3-4. In one example, the deformable segment 216 is substantially parallel to the side wall 212 of the catheter 202 in the first position. The deformable segment 216 is configured to project away from the side wall 212 of the catheter 202 towards a midline of the lumen 208 in a second position to at least partially obstruct the lumen 208 of the catheter 202 such that the first opening 210A is accessible via the lumen 108, as shown in FIGS. 5-6.

In one example, as shown in FIGS. 3-4, the apparatus 200 may further include a guide wire 218 positioned in the lumen 208 in the first position. In the example shown in FIG. 3, the guide wire 218 is shown as a through wire extending from the first end 204 of the catheter 202 to the second end 206 of the catheter 202. In the example shown in FIGS. 5-6, the guide wire 218 is configured to contact the deformable segment 216 in the second position such that the guide wire 218 is thereby directed through the first opening 210A in the side wall 212 of the catheter 202 in the second position. In particular, in the second position the deformable segment 216 extends away from the side wall 212 of the catheter 202 and into the lumen 208, and the guide wire 218 contacts an angled surface 220 of the deformable segment 216 to thereby direct the guide wire 218 through the first opening 210A and out of the catheter 202, as shown in FIG. 5. As such, the guide wire 218 is able to more easily access arterial branches or side branches out of the one or more openings 210A-210C when the deformable segment 216 is transitioned to the second position.

The deformable segment 216 may take a variety of forms, each of which act to divert the guide wire 218 out of the first opening 210A in the second position. In one example, as shown in FIG. 5, the deformable segment 216 includes a shaft 224 having a first end 226 and a second end 228. In such an example, the second end 228 of the shaft 224 is coupled to an interior surface 230 of the lumen 208 of the catheter 202, and the first end 226 of the shaft 224 is configured to project away from the side wall 212 of the catheter 202 towards the midline of the lumen 208 to at least partially obstruct the lumen 208 of the catheter 202 in the second position. In another example, as shown in FIG. 4, the deformable segment 216 includes a sheath 232 including a slit 234 defining an outline of the deformable segment 216 and permitting access to the lumen 208 via the first opening 210A in the second position. In such an example, the sheath 232 includes a member 236 that covers the first opening 110A in the first position and projects away from the side wall 212 of the catheter 202 and into the lumen 208 in the second position to thereby direct the guide wire 218 through the first opening 110A in the side wall 212 of the catheter 202. Other examples are possible as well.

In one example, the deformable segment 216 includes a shape memory material that is biased to have an arrangement projecting away from the side wall 212 of the catheter 202 towards the midline of the lumen 208 in a relaxed condition. In another example, the deformable segment 216 is configured to transition from the first position to the second position in response to an external force on an outer surface 236 of the catheter 202. For example, the deformable segment 216 may be configured to transition from the first position to the second position in response to a pinching of the outer surface 236 of the catheter 202. In another example, the deformable segment 216 is configured to transition from the first position to the second position in response to a bending of the catheter 202. In yet another example, the deformable segment 216 is configured to transition from the first position to the second position in response to introducing a diverter 116, such as an elongated shaft or blunted needle as examples, through the first opening 210A from an exterior of the catheter 202 and into the lumen 208 of the catheter 202. Each of the above examples may break a connection between a first end 238 of the deformable segment 216 and the side wall 212 of the catheter 202, to thereby transition the deformable segment 216 from the first position to the second position to enable the first end 238 to project away from the side wall 202 of the catheter 202 towards a midline of the lumen 208 to at least partially obstruct the lumen 208 of the catheter 202 such that the first opening 210A is accessible via the lumen 208.

In one example, the first opening 210A is positioned between about 10 mm to about 240 mm from the first end 204 of the catheter 202. In another example, the apparatus 200 further includes a second opening 210B of the one or more openings 210A-210C positioned between the first opening 210A and the second end 206 of the catheter 202. In such an example, the apparatus 200 may further include a second deformable segment 240 positioned in the lumen 208. The second deformable segment 240 positioned at least partially over the second opening 210B in a first position, and the second deformable member 240 is configured to project away from the side wall 212 of the catheter 202 towards a midline of the lumen 208 to at least partially obstruct the lumen 208 in a second position of the catheter 202 such that the second opening 210B is accessible via the lumen 208. The second deformable segment 240 may be substantially parallel to the side wall 212 of the catheter 202 in the first position. The first opening 210A may be positioned between about 10 mm to about 50 mm from the first end 204 of the catheter 202, and the second opening 210B may be positioned between about 30 mm to about 240 mm from the first end 204 of the catheter 202.

In yet another example, the apparatus 200 further includes a third opening 210C of the one or more openings 210A-210C positioned between the second opening 210B and the second end 206 of the catheter 202. In such an example, the apparatus 202 further includes a third deformable member 242 positioned in the lumen 208. The third deformable member 242 is positioned at least partially over the third opening 210C in a first position. The third deformable member 242 is configured to project away from the side wall 212 of the catheter 202 towards a midline of the lumen 208 in a second position to at least partially obstruct the lumen 208 of the catheter 202 such that the third opening 210C is accessible via the lumen 208. The third deformable member 242 may be substantially parallel to the side wall 212 of the catheter 202 in the first position. The first opening 210A may be positioned between about 10 mm to about 50 mm from the first end 204 of the catheter 202, the second opening 21B may be positioned between about 30 mm to about 100 mm from the first end 204 of the catheter 202, and the third opening 210C may be positioned between about 50 mm to about 240 mm from the first end 204 of the catheter 202.

In one example, each of the first opening 210A, the second opening 210B, and the third opening 210C have the same diameter. In another example, each of the first opening 210A, the second opening 210B, and the third opening 210C have different diameters. The first opening 210A, the second opening 210B, and the third opening 210C gives a user the ability to determine which one (if any) of the multiple deformable segments 216, 240, 242 and corresponding openings 210A-210C should be transitioned from the first position to the second position prior to inserting the apparatus 200 into a patient, depending on the particular use case.

Each of the above examples of transitioning the deformable segments 216, 240, 242 from the first position to the second position may occur prior to deployment of the apparatus 200. As such, the transition of the deformable segments 216, 240, 242 from the first position to the second position may occur when the apparatus 100 is ex vivo. Each of the deformable segments 216, 240, 242 may be configured to be permanently deformed to the second position in response to an external force applied to an outer surface 236 of the catheter 202.

In one example, the liner 214 comprises a perforated material positioned over the first opening 210A in the catheter 202. In such an example, the perforated material may comprise a gel, a mesh, a cloth, or polytetrafluoroethylene (PTFE). In one example, the liner 210 may include a longitudinal or transverse slit 234 in the perforated material such that the longitudinal or transverse slit 234 is sealed in the first position and deforms to form an opening 210A-210C in the second position. The longitudinal or transverse slit 234 may define the deformable segment 216 and permit access to the lumen 208 via the first opening 210A in the second position.

The apparatus 200 may further include a balloon 244 coupled to the first end 204 of the catheter 202. In one example, such a balloon 244 may be a sail balloon. In operation, this arrangement may permit the balloon 244 to be advanced (e.g., floated) to the target lumen by blood flow and thereby advance the apparatus 200. The operator may observe the advancement on a fluoroscopic image and may stop advancement once the apparatus 200 is disposed within the desired arterial configuration.

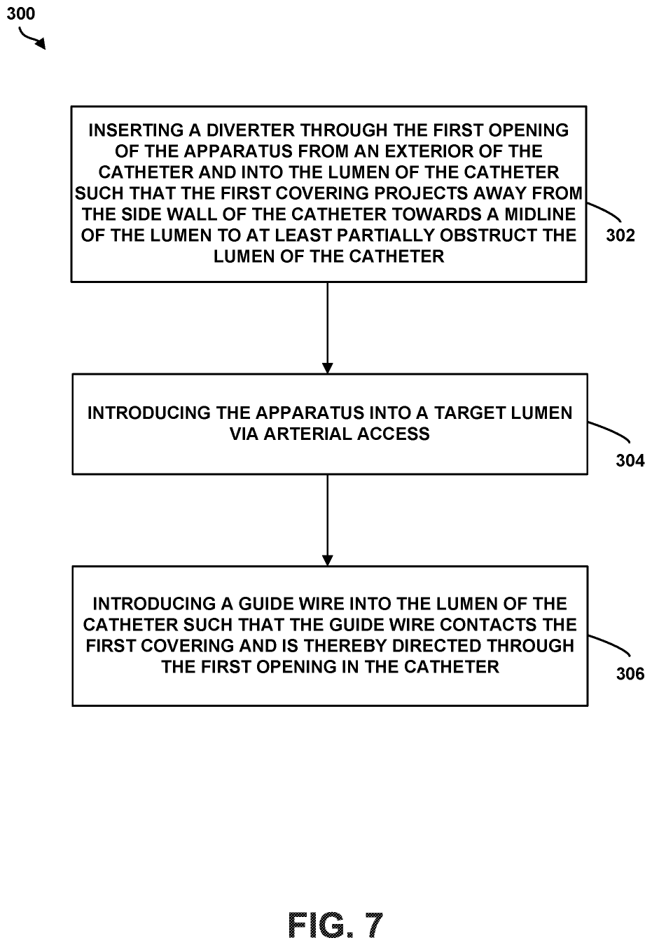

FIG. 7 is a simplified flow chart illustrating a method 300 according to an exemplary embodiment. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 302, the method 300 includes inserting a diverter 116 through the first opening 110A of the apparatus 100 from an exterior of the catheter 102 and into the lumen 108 of the catheter 102 such that the first covering 114A projects away from the side wall 112 of the catheter 102 towards a midline of the lumen 108 to at least partially obstruct the lumen 108 of the catheter 102. As discussed above, the diverter 116 may be inserted into the first opening 110A when the apparatus 100 is ex vivo. The first covering 114A may be configured to be permanently deformed to the second position in response to a force applied by the diverter 116 to the first covering 114A. At block 304, the method 300 includes introducing the apparatus 100 into a target lumen via arterial access. At block 306, the method 300 includes introducing a guide wire 118 into the lumen 108 of the catheter 102 such that the guide wire 118 contacts the first covering 114A and is thereby directed through the first opening 110A in the catheter 102.

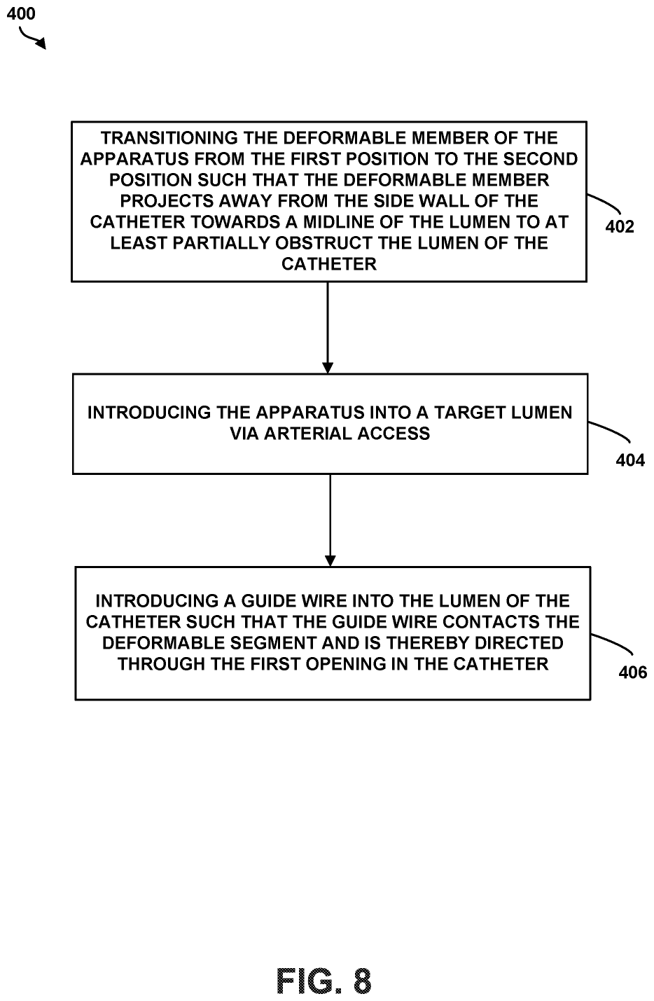

FIG. 8 is a simplified flow chart illustrating another method 400 according to an exemplary embodiment. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 402, the method 400 includes transitioning the deformable member 216 of the apparatus 200 from the first position to the second position such that the deformable member 216 projects away from the side wall 212 of the catheter 202 towards a midline of the lumen 208 to at least partially obstruct the lumen 208 of the catheter 202. As discussed above, the transitioning of the deformable member 216 from the first position to the second position may occur when the apparatus 100 is ex vivo. The deformable member 216 may be configured to be permanently deformed to the second position in response to a force applied to the catheter 202. At block 404, the method 400 includes introducing the apparatus 200 into a target lumen via arterial access. At block 406, the method 400 includes introducing a guide wire 218 into the lumen 208 of the catheter 202 such that the guide wire 218 contacts the deformable segment 216 and is thereby directed through the first opening 210A in the catheter 202.

In one example, transitioning the deformable segment 216 from the first position to the second position includes applying an external force on an outer surface 236 of the catheter 202. For example, the deformable segment 216 may be configured to transition from the first position to the second position in response to a pinching of the outer surface 236 of the catheter 202. In another example, transitioning the deformable segment 216 from the first position to the second position includes bending the catheter 202. In yet another example, transitioning the deformable segment 216 from the first position to the second position includes introducing a diverter 116, such as an elongated shaft or a blunted needle as one example, through the first opening 210A from an exterior of the catheter 202 and into the lumen 208 of the catheter 202.

Each of the above examples may break a connection between a first end 238 of the deformable segment 216 and the side wall 212 of the catheter 202, to thereby transition the deformable segment 216 from the first position to the second position to enable the first end 238 of the deformable segment 216 to project away from the side wall 212 of the catheter 202 towards a midline of the lumen 208 to at least partially obstruct the lumen 208 of the catheter 202 such that the first opening 210A is accessible via the lumen 208. The transitioning of the deformable segment 216 from the first position to the second position may be performed ex vivo prior to insertion of the apparatus 200 into the patient. As described above, there may be multiple deformable segments 216, 240, 242 covering multiple openings 210A-210C in the side wall 212 of the catheter 202. In such an example, a medical professional may determine which one (if any) of the multiple deformable segments 216, 240, 242 should be transitioned from the first position to the second position prior to inserting the apparatus 200 into a patient, depending on the particular use case.

The method 400 may further include the steps of inflating a balloon 244 coupled to the first end 204 of the catheter 202, and advancing the first end 204 of the catheter 202, via the balloon 244, to the target lumen. In this embodiment, the inflated balloon 244 may be acted upon by blood flow to deliver the apparatus 200 to the target lumen.

In the above description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts were described in conjunction with specific examples, it will be understood that these examples are not intended to be limiting.

1. An apparatus comprising:
a catheter having a first end and a second end, wherein the catheter defines a lumen extending from the first end to the second end;
one or more openings positioned in a side wall of the catheter between the first end and the second end of the catheter; and
one or more coverings positioned over the one or more openings in the catheter in a first position, wherein a diverter is configured to contact a first covering of the one or more coverings such that the first covering transitions from the first position where the first covering is positioned over a first opening of the one or more openings to a second position where the first covering projects away from a side wall of the catheter towards a midline of the lumen to at least partially obstruct the lumen of the catheter such that the first opening of the one or more openings is accessible via the lumen.

2. The apparatus of claim 1, further comprising a guide wire positioned in the lumen when the first covering is in the first position.

3. The apparatus of claim 1, wherein the guide wire is configured to contact the first covering when the first covering is in the second position such that the guide wire is thereby directed through the first opening in the side wall of the catheter.

4. The apparatus of claim 1, wherein the first opening is positioned between about 10 mm to about 240 mm from the first end of the catheter.

5. The apparatus of claim 1, wherein a second opening of the one or more openings is positioned between the first opening and the second end of the catheter, wherein a second covering of the one or more coverings is positioned over the second opening when the second covering is in the first position, wherein the second opening is configured to receive the diverter such that the second covering projects away from the side wall of the catheter towards the midline of the lumen to at least partially obstruct the lumen of the catheter such that the second opening is accessible via the lumen when the second covering is in the second position.

6. The apparatus of claim 5, wherein the first opening is positioned between about 10 mm to about 50 mm from the first end of the catheter, and wherein the second opening is positioned between about 30 mm to about 240 mm from the first end of the catheter.

7. The apparatus of claim 5, wherein a third opening of the one or more openings is positioned between the second opening and the second end of the catheter, wherein a third covering of the one or more coverings is positioned over the third opening when the third covering is in the first position, wherein the third opening is configured to receive the diverter such that the third covering projects away from the side wall of the catheter towards the midline of the lumen to at least partially obstruct the lumen of the catheter such that the third opening is accessible via the lumen when the third covering is in the second position.

8. The apparatus of claim 7, wherein the first opening is positioned between about 10 mm to about 50 mm from the first end of the catheter, wherein the second opening is positioned between about 30 mm to about 100 mm from the first end of the catheter, and wherein the third opening is positioned between about 50 mm to about 240 mm from the first end of the catheter.

9. The apparatus of claim 1, wherein each of the one or more coverings are configured to be permanently deformed to the second position in response to a force applied by the diverter.

10. The apparatus of claim 1, wherein the one or more coverings comprise a perforated material including a gel, a mesh, a cloth, or polytetrafluoroethylene (PTFE).

11. The apparatus of claim 1, wherein the diverter comprises an elongated shaft positionable through the first opening from an exterior of the catheter and into the lumen of the catheter.

12. An apparatus comprising:
a catheter having a first end and a second end, wherein the catheter defines a lumen extending from the first end to the second end;
one or more openings positioned in a side wall of the catheter between the first end and the second end of the catheter; and
a liner having a deformable segment positioned in the lumen, wherein the deformable segment is positioned at least partially over a first opening of the one or more openings when the deformable segment is in a first position, and wherein the deformable segment is configured to project away from the side wall of the catheter towards a midline of the lumen in a second position to at least partially obstruct the lumen of the catheter such that the first opening is accessible via the lumen.

13. The apparatus of claim 12, further comprising a guide wire positioned in the lumen when the deformable segment is in the first position.

14. The apparatus of claim 12, wherein the guide wire is configured to contact the deformable segment when the deformable segment is in the second position such that the guide wire is thereby directed through the first opening in the side wall of the catheter.

15. The apparatus of claim 12, wherein the liner comprises a shaft having a first end and a second end, wherein the second end of the shaft is coupled to an interior surface of the lumen of the catheter, and wherein the deformable segment comprises the first end of the shaft and is configured to project away from the side wall of the catheter towards the midline of the lumen to at least partially obstruct the lumen of the catheter in the second position.

16. The apparatus of claim 12, wherein the liner comprises a sheath including a slit defining an outline of the deformable segment and that permits access to the lumen via the first opening in the second position.

17. The apparatus of claim 12, wherein the deformable segment comprises a shape memory material and is biased to have an arrangement projecting away from the side wall of the catheter towards the midline of the lumen in a relaxed condition.

18.-30. (canceled)

31. A method comprising:
inserting a diverter through the first opening of the apparatus according to claim 1 from an exterior of the catheter and into the lumen of the catheter such that the first covering projects away from the side wall of the catheter towards a midline of the lumen to at least partially obstruct the lumen of the catheter;
introducing the apparatus into a target lumen via arterial access; and
introducing a guide wire into the lumen of the catheter such that the guide wire contacts the first covering and is thereby directed through the first opening in the catheter.

32. A method comprising:
transitioning the deformable member of the apparatus according to claim 12 from the first position to the second position such that the deformable member projects away from the side wall of the catheter towards a midline of the lumen to at least partially obstruct the lumen of the catheter;
introducing the apparatus into a target lumen via arterial access; and
introducing a guide wire into the lumen of the catheter such that the guide wire contacts the deformable segment and is thereby directed through the first opening in the catheter.

33.-36. (canceled)
